(19) United States
(12) Patent Application Publication
(10) Pub. No.: US 2008/0153128 A1
(43) Pub. Date: Jun. 26, 2008

(54) Cell-Free Protein Synthesis

(75) Inventors: Dong-Myung Kim, Daejeon (KR); Jin-Ho Ahn, Seoul (KR); Cha-Yong Choi, Seoul (KR)

Correspondence Address: CLARK & ELBING LLP, 101 FEDERAL STREET, BOSTON, MA, 02110, US

(73) Assignee: ISU ABXIS CO., LTD., Seoul (KR)

(21) Appl. No.: 11/877,377

(22) Filed: Oct. 23, 2007

Related U.S. Application Data
(60) Provisional application No. 60/862,601, filed on Oct. 23, 2006.

(51) Int. Cl. C12P 21/06

(52) U.S. Cl. 435/68.1

(57) ABSTRACT

The present invention relates to a method for synthesizing a protein in a cell-free synthesis system, which comprises the steps of: (a) amplifying a DNA molecule encoding the protein to prepare linear DNA molecules with a stem and loop sequence at their terminus, wherein the stem and loop sequence comprises a nucleotide sequence inducing the formation of a stem and loop structure at the 3′-end of transcripts of the amplified DNA molecules to prevent degradation of the transcripts; and (b) contacting the amplified DNA molecules to a lysate of cells with deficient endoribonuclease E activity, thereby producing the protein in the cell-free synthesis system.

### CROSS REFERENCE TO RELATED APPLICATIONS

This application claims benefit under 35 U.S.C. § 119(e) of U.S. Ser. No. 60/862,601, filed Oct. 23, 2006, the contents of which are incorporated herein by reference.

### BACKGROUND OF THE INVENTION

1. Field of the Invention

The present invention relates to a method for synthesizing a protein in a cell-free synthesis system.

2. Description of the Related Art

Cell-free protein synthesis is now actively considered as an attractive alternative to conventional in vivo gene expression technology [1]. As cell-free protein synthesis does not necessitate cell growth, multiple proteins can be prepared with minimal processing time and laboratory set-up. In particular, as the demand for high-throughput protein expression increases, due to recent progress made in many genome sequencing projects, it is expected that the application of cell-free synthesis will be rapidly expanded [2, 3].

However, in most of the current protocols for cell-free synthesis, protein synthesis is directed by plasmid templates and it is still necessary to grow cells for the cloning and amplification of the DNAs, which off-sets the merit of using a cell-free system. The time- and labor-intensive steps required for plasmid preparation could be avoided if the template DNAs were able to be prepared by PCR. The rapid preparation of the expression templates based on PCR methods would enable the discrepancy between the throughputs of template preparation and their translation to be overcome. However, even though several reports describing the cell-free expression of PCR products have already been published, generally, the efficiency of cell-free synthesis is very low compared to the reactions utilizing plasmid templates. This is mainly due to the rapid decay of the linear templates by the exonucleases present in the cell-free extract [4, 5]. Disruption of the exonuclease genes on the chromosome of Escherichia coli can be considered for the stable maintenance of linear DNAs in a cell-free extract [6]. However, exonuclease-deficient stains generally show significant defects in their growth. Also, in our experience, the extracts prepared from these mutant strains exhibit a serious reduction in their translational activity. Therefore, it seems very difficult or even impossible to prevent the rapid decay of the linear templates in cell-free extracts, and this makes the importance of mRNA stability augmented in improving the productivity of PCR-based cell-free protein synthesis. Although the life span of mRNA in E. coli extracts is very short, during cell-free protein synthesis based on a plasmid template, the continued transcription of the mRNA molecules compensates for the rapid turn-over of the mRNA molecules. However, since the linear templates can only provide mRNA transcripts for a limited range of incubation times, the level of protein expression is more dependent on the length of time that the transcripts remain functionally intact in the cell-free extract.

Throughout this application, various patents and publications are referenced, and citations are provided in parentheses. The disclosure of these patents and publications in their entities are hereby incorporated by references into this application in order to more fully describe this invention and the state of the art to which this invention pertains.

### SUMMARY OF THE INVENTION

The present inventors have made intensive researches to develop a novel approach for synthesizing proteins in a cell-free synthesis system. As a result, we verified that the life span of mRNA is remarkably extended when its 3′-end is made to form a stem-loop structure and the cell-free protein synthesis is conducted in an extract lacking endoribonuclease E (RNase E) activity. This enhanced stability of the mRNA, in turn, leads to highly efficient expression of proteins at comparable yields to those of reactions utilizing plasmid templates.

Accordingly, it is an object of this invention to provide a method for synthesizing a protein in a cell-free synthesis system.

Other objects and advantages of the present invention will become apparent from the detailed description to follow taken in conjugation with the appended claims and drawings.

### BRIEF DESCRIPTION OF THE DRAWINGS

FIGS. 1A and 1B represent the effect of 3′-tail secondary structure on translational efficiency in a coupled transcription/translation system. 0.5 μg of each PCR-amplified DNA was added. The reaction mixture was incubated at 37° C. for 2 hours. (FIG. 1A) Fifteen microliter samples were withdrawn and the [¹⁴C]-leucine-labeled radioactivity of the protein was measured as described in “Materials and Methods.” (FIG. 1B) The fluorescent activity of EGFP was measured after the reaction using a fluorescence spectrometer at an excitation wavelength of 488 nm and an emission wavelength of 507 nm. T7T represents the natural T7 terminator.

FIG. 2 demonstrates the effect of 3′ stem-loop on mRNA stability during protein synthesis. At the indicated time point, five microliter samples were removed from the reaction mixture being incubated to examine the mRNA kinetics. The samples isolated were analyzed by denaturing electrophoresis on a 2% formaldehyde agarose gel and the RNA molecules were stained with ethidium bromide.

FIG. 3 represents the effect of the length of stem structure on the efficacy of the T7 terminator sequence to stimulate protein synthesis. By PCR, T7 terminator sequences with varying length of stem structure were added to the 3′ end of EGFP sequence. The numbers indicate the number of base pairs in the stem structure. For instance, T7-15 represents the natural 17 terminator sequence whose stem structure consists of 15 base pairs. T7-15 was amplified using the T7 terminator primer as a backward primer of Table 1 or the following primer sequence: CAAAAAACCCCTCAAGACCCGTTTAGAGGCCCCAAGGGGTTATGCTAGTTATTGCT. Resulting PCR products were used as the templates for cell-free protein synthesis as described in Materials and Methods.

FIGS. 4A-4E represent the effect of T7 terminator stem-loop and RNase E defective (rne 131 mutation) cell extract on the translational efficiency. Fifteen microliter samples were withdrawn and the [¹⁴C]-leucine-labeled radioactivity of the protein was measured.

FIGS. 5A-5C represent the comparison of the protein syntheses with linear or circular DNAs as the templates. A reaction mixture for cell-free protein synthesis was prepared using the rne 131 S30 extract as described in the Materials and Methods. FIG. 5A: After 2 hours of incubation, 2 μl of the reaction mixture was loaded on tricine SDS-PAGE and stained with Coomassie Brilliant Blue (CBB). Cell-free expressed proteins are indicated by asterisks on the right side of the protein bands. ‘C’ and ‘L’ refer to the circular plasmid and linear PCR-amplified DNA, respectively. FIG. 5B: At the indicated time points during the incubation of reaction mixture, 5 μl of the reaction mixture was isolated and analyzed by denaturing electrophoresis on a 2% formaldehyde agarose gel and the RNA molecules were stained with ethidium bromide. FIG. 5C: time-courses of intact mRNA were compared between the linear template and plasmid templates encoding different proteins. The linear templates were prepared by PCR with the backward primers containing the T7-terminator sequence. After 30 minutes of incubation, 5 μl of the reaction mixture was isolated and analyzed by denaturing electrophoresis on a 2% formaldehyde agarose gel. EGFP: enhanced green fluorescence protein, DHFR: dihydrofolate reductase, EPO: erythropoietin, UK: urokinase, CAT: chloramphenicol acetyl transferase.

### DETAILED DESCRIPTION OF THIS INVENTION

In one aspect of this invention, there is provided a method for synthesizing a protein in a cell-free synthesis system, which comprises the steps of:

(a) amplifying a DNA molecule encoding the protein to prepare linear DNA molecules with a stem and loop sequence at their terminus, wherein the stem and loop sequence comprises a nucleotide sequence inducing the formation of a stem and loop structure at the 3′-end of transcripts of the amplified DNA molecules to prevent degradation of the transcripts; and

(b) contacting the amplified DNA molecules to a lysate of cells with deficient endoribonuclease E activity, thereby producing the protein in the cell-free synthesis system.

The present inventors have made intensive researches to develop a novel approach for synthesizing proteins in a cell-free synthesis system. As a result, we verified that the life span of mRNA is remarkably extended when its 3′-end is made to form a stem-loop structure and the cell-free protein synthesis is conducted in an extract lacking endoribonuclease E (RNase E) activity. This enhanced stability of the mRNA, in turn, leads to highly efficient expression of proteins at comparable yields to those of reactions utilizing plasmid templates.

In the first step of the present method, the DNA molecule encoding a protein of interest is amplified.

The amplification of nucleic acid molecules has been performed in accordance with a multitude of amplification reactions known to one of skill in the art, including polymerase chain reaction (hereinafter referred to as PCR) (U.S. Pat. Nos. 4,683,195, 4,683,202, and 4,800,159), reverse transcription-polymerase chain reaction (hereinafter referred to as RT-PCR) (Sambrook, J. et al., Molecular Cloning. A Laboratory Manual, 3rd ed. Cold Spring Harbor Press (2001)), the methods of Miller, H. I. (WO 89/06700) and Davey, C. et al. (EP 329,822), ligase chain reaction (LCR), Gap-LCR (WO 90/01069), repair chain reaction (EP 439,182), transcription-mediated amplification CTMA) (WO 88/10315), self sustained sequence replication (WO 90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909 and 5,861,245), nucleic acid sequence based amplification (NASBA) (U.S. Pat. Nos. 5,130,238, 5,409,818, 5,554,517, and 6,063,603), strand displacement amplification and loop-mediated isothermal amplification (LAMP), but not limited to. Other amplification methods that may be used are described in, U.S. Pat. Nos. 5,242,794, 5,494,810, 4,988,617 and in U.S. Ser. No. 09/854,317.

According to a preferred embodiment, the amplification is carried out by primer-based nucleic acid amplification methods including the methods of Miller, H. I. (WO 89/06700) and Davey, C. et al. (EP 329,822), Ligase Chain Reaction (L C R, Wu, D. Y et al., Genomics 4:560 (1989)), Polymerase Ligase Chain Reaction (Barany, PCR Methods and Applic., 1:5-16 (1991)), Gap-LCR (WO 90/01069), Repair Chain Reaction (EP 439,182), 3SR (Kwoh et al., PNAS, USA, 86:1173(1989)) and NASBA (U.S. Pat. No. 5,130,238), but not limited to.

Where a mRNA is employed as starting material, a reverse transcription step is necessary prior to performing annealing step, details of which are found in Joseph Sambrook, et al., Molecular Cloning, A Laboratory Manual, Cold Spring Harbor Laboratory Press, Cold Spring Harbor, N.Y. (2001); and Noonan, K. F. et al., Nucleic Acids Res. 16:10366 (1988)). For reverse transcription, an oligonucleotide dT primer hybridizable to poly A tail of mRNA is used. The oligonucleotide dT primer is comprised of dTMPs, one or more of which may be replaced with other dNMPs so long as the dT primer can serve as primer. Reverse transcription can be done with reverse transcriptase that has RNase H activity. If one uses an enzyme having RNase H activity, it may be possible to omit a separate RNase H digestion step by carefully choosing the reaction conditions.

The most preferable amplification reaction in the present invention is a polymerase chain reaction (hereinafter referred to as “PCR”), which is based on repeated cycles of denaturation of double-stranded DNA, followed by oligonucleotide primer annealing to the DNA template, and primer extension by a DNA polymerase (Mullis et al. U.S. Pat. Nos. 4,683,195, 4,683,202, and 4,800,159)

The process for amplifying the DNA molecule by primer annealing, primer extending and denaturing is well known to those of skill in the art.

Suitable annealing or hybridization conditions may be routinely determined by optimization procedures. Conditions such as temperature, concentration of components, hybridization and washing times, buffer components, and their pH and ionic strength may be varied depending on various factors, including the length and GC content of primer and target nucleotide sequence. The detailed conditions for hybridization can be found in Joseph Sambrook, et al., Molecular Cloning, A Laboratory Manual, Cold Spring Harbor Laboratory Press, Cold Spring Harbor, N.Y. (2001); and M. L. M. Anderson, Nucleic Acid Hybridization, Springer-Verlag New York Inc. N.Y. (1999).

Where the DNA molecule as templates is typically double-stranded, it is preferred to render the two strands into a single-stranded or partially single-stranded form. Methods known to separate strands includes, but not limited to, heating, alkali, formamide, urea and glycoxal treatment, enzymatic methods (e.g., helicase action), and binding proteins. For instance, strand separation can be achieved by heating at temperature ranging from 80° C. to 105° C. General methods for accomplishing this treatment are provided by Joseph Sambrook, et al., Molecular Cloning, A Laboratory Manual, Cold Spring Harbor Laboratory Press, Cold Spring Harbor, N.Y. (2001).

Conditions of nucleic acid annealing suitable for forming such double stranded structures are described by Joseph Sambrook, et al., Molecular Cloning, A Laboratory Manual, Cold Spring Harbor Laboratory Press, Cold Spring Harbor, N.Y. (2001) and Haymes, B. D., et al., Nucleic Acid Hybridization, A Practical Approach, IRL Press, Washington, D.C. (1985).

A variety of DNA polymerases can be used in the extension reactions, which includes “Klenow” fragment of E. coli DNA polymerase I, a thermostable DNA polymerase, and bacteriophage T7 DNA polymerase. Preferably, the polymerase is a thermostable DNA polymerase, which may be obtained from a variety of bacterial species, including Thermus aquaticus (Taq), Thermus thermophilus (Tth), Thermus filiformis, Thermis flavus, Thermococcus literalis, and Pyrococcus furiosus (Pfu). When a polymerization reaction is being conducted, it is preferable to provide the components required for such reaction in excess in the reaction vessel. Excess in reference to components of the extension reaction refers to an amount of each component such that the ability to achieve the desired extension is not substantially limited by the concentration of that component. It is desirable to provide to the reaction mixture an amount of required cofactors such as Mg²⁺, dATP, dCTP, dGTP, and dTTP in sufficient quantity to support the degree of the extension desired.

Annealing or hybridization is performed under stringent conditions that allow for specific binding between the primer and the template nucleic acid. Such stringent conditions for annealing will be sequence-dependent and varied depending on environmental parameters.

In the most preferable embodiment, the amplification is performed in accordance with PCR (polymerase chain reaction), which is disclosed in U.S. Pat. Nos. 4,683,195, 4,683,202, and 4,800,159.

In the present method, the amplification is intended to introduce a stem and loop sequence into the amplified DNA molecules, wherein the stem and loop sequence comprises a nucleotide sequence inducing the formation of a stem and loop structure at the 3′-end of transcripts of the amplified DNA molecules. Such introduction may be accomplished using primers having stem and loop sequences or using templates having genes coding for proteins of interest as well as flanking stem and loop sequences as exemplified in Examples.

The nucleotide sequences capable of stem and loop structures are well known to those skilled in the art. For instance, an inverted repeat sequence is able to form a stem and loop structure for preventing degradation of mRNA.

According to a preferred embodiment, the stem of the stem and loop structure has 5-30 base pairs, more preferably, 15-20 base pairs.

According to a preferred embodiment, the stem of the stem and loop structure is a transcription termination sequence such as T7 termination sequence, SP6 termination sequence (Dobbins A T et al., Complete genomic sequence of the virulent Salmonella bacteriophage SP6, (2004), 186, 1933-1944), T3 terminator sequence (Sengupta D et al, Relative efficiency of utilization of promoter and termination sites by bacteriophage T3 RNA polymerase, (1989), 264, 14246-14255), and E. coli termination sequence like Rho-independent terminator (Abe H et al., Regulation of intrinsic terminator by translation in Escherichia coli: transcription termination at a distance downstream, (1999), 4, 87-97).

According to a preferred embodiment, the stem and loop structure has the nucleotide sequence selected from the group consisting of SEQ ID NOs:12-19.

More preferably, the stem and loop structure useful in this invention the nucleotide sequence selected from the group consisting of SEQ ID NOs:14-18. Most preferably, the stem and loop structure is set forth in SEQ ID NOs:15 or 16.

The proteins encoded by the DNA molecule used in this invention may include, but not limited to, hormones, hormone analogues, enzymes, enzyme inhibitors, signal transduction proteins or fragments thereof, antibodies or fragments thereof, single chain antibodies, binding proteins or fragments thereof, peptides, antigens, adhesive proteins, structural proteins, regulatory proteins, toxin proteins, cytokines, transcription regulatory proteins, blood clotting proteins and plant defense-inducing proteins.

Preferably, the protein encoded by the DNA molecule used in this invention is enhanced green fluorescence protein, dihydrofolate reductase, erythropoietin, urokinase or chloramphenicol acetyltransferase, more preferably, enhanced green fluorescence protein, dihydrofolate reductase, erythropoietin or urokinase, most preferably, dihydrofolate reductase, erythropoietin or urokinase.

Afterwards, the amplified DNA molecules are incubated with a lysate of cells with deficient endoribonuclease E (RNase E) activity to produce a protein of interest.

The most striking feature of the present cell-free synthesis system is to utilize a lysate of cells with deficient endoribonuclease E (RNase E) activity. The lysate lacking RNase E activity is responsible for dramatic increase in protein productivity.

The lysate used in this invention may be obtained from various prokaryotic or eukaryotic cells lacking RNase E activity. For example, the lysate may be prepared from E. coli, plants of the family Poaceae such as wheat, barley, rice and corn, and rabbit reticulocytes.

Preferably, the lysate is derived from E. coli, rabbit reticulocyte or wheat germ. More preferably, the lysate used in this invention is obtained from E. coli with deficient RNase E activity, most preferably, E. coli rne 131 strain with deficient RNase E activity. The wild type of E. coli RNase E consists of 1061 amino acid residues. The E. coli me 131 strain of which genotype is described as F⁻ ompT hsdS_{B} (r_{B}⁻m_{B}⁻) gal dcm me 131 (DE3), has a mutant type of RNase E with deleted 477 amino acid residues at its C-terminal and is commercially available from Invitrogen, Inc. (USA).

It is preferred that the amount of the lysate for the protein synthesis ranges from 10 (v/v) % to 60 (v/v) %, particularly preferably 20 (v/v) % to 40 (v/v) %.

The cell-free synthesis reaction for this invention may be constructed according to conventional technology known to one of skill in the art.

The cell-free synthesis reaction used in the step (b) generally comprises at least one selected from the group consisting of an ammonium salt, DTT (dithiothreitol), rNTP (ATP, GTP, UTP and CTP), creatine phosphate, creatine kinase, amino acid components, tRNA, folinic acid and a buffer. Preferably, the step (b) is carried out using the above all components.

For example, the ammonium salt in the synthesis reaction solution includes ammonium chloride, ammonium nitrate, ammonium sulfate, ammonium carbonate and ammonium acetate. Among them, ammonium acetate is preferably used. The content of the ammonium salt in the synthesis reaction is not particularly limited. For example, the ammonium salt such as ammonium acetate is contained at preferably 10 mM to 200 mM, more preferably 60 mM to 100 mM.

The DDT is formulated for the purpose of preventing oxidation and is contained at preferably 0.1 mM to 10 mM, more preferably 1.0 mM to 3 mM, in the synthesis reaction.

ATP in the synthesis reaction is contained at preferably 0.1 mM to 10 mM, more preferably 0.5 mM to 3 mM. GTP, UTP and CTP in the synthesis reaction solution is contained at preferably 0.1 mM to 5 mM, more preferably 0.5 mM to 2 mM.

Creatine phosphate in the synthesis reaction solution is a component for continuously synthesizing proteins and is formulated for the purpose of regenerating ATP and GTP. Creatine phosphate is contained at preferably 10 mM to 200 mM, more preferably 30 mM to 100 mM.

Creatine kinase in the reaction solution is a component for continuously synthesizing proteins and is formulated for the purpose of regenerating ATP and GTP in concert with creatine phosphate. Creatine kinase is contained at preferably 0.05-2 units/mL, more preferably 0.1-1 unit/mL.

The amino acid components contained in the synthesis reaction solution are at least 20 amino acids, that is, valine, methionine, glutamic acid, alanine, leucine, phenylalanine, glycine, proline, isoleucine, tryptophan, asparagine, serine, threonine, histidine, aspartic acid, tyrosine, lysine, glutamine, cysteine, and arginine. The amino acid components usually comprise approximately equal amounts of the amino acids of these types. In the present invention, the amino acid components are contained at preferably 0.1 mM to 10 mM, more preferably 0.5 mM to 3 mM.

The tRNA in the synthesis reaction solution comprises approximately equal amounts of tRNAs of types corresponding to the 20 amino acids described above. In the present invention, the tRNA is contained at preferably 0.01 mg/mL to 2 mg/mL, more preferably 0.1 mg/mL to 1 mg/mL.

Folinic acid in the synthesis reaction is contained at preferably 1-100 mg/mL, more preferably 10-60 mg/mL.

The buffer is formulated for the purpose of imparting buffer capacity to the synthesis reaction solution and, for example, preventing the denaturation of the extract and/or reaction products attributed to rapid changes in the pH of the reaction solution caused by the addition of an acidic or basic substance. Such a buffer is not particularly limited. For example, Hepes-KOH, Tris-HCl, acetic acid-sodium acetate, citric acid-sodium citrate, phosphoric acid, boric acid, MES, and PIPES can be used. Preferably, the buffer used keeps the pH of the synthesis reaction solution at 4 to 10, more preferably 7.5 to 8.5. Among the buffers described above, HEPES-KOH (pH 7.5 to 8.5) is particularly preferably used. The content of the buffer in the synthesis reaction solution is not particularly limited and is preferably 10 mM to 200 mM, more preferably 40 mM to 80 mM.

The amplified DNA molecules as templates are contained at 1-100 μg/mL, more preferably 20-60 μg/mL.

According to a preferred embodiment, the step (b) is carried out by contacting the amplified DNA molecules to the lysate of cells with deficient RNase E activity in the presence of 10 mM to 200 mM ammonium salt, 0.1 mM to 10 mM DIT, 0.1 mM to 5 mM ATP, 0.1 mM to 5 mM GTP, UTP and CTP, 10 mM to 200 mM creatine phosphate, 0.005-2 units/mL creatine kinase, 0.1 mM to 10 mM amino acid components, 0.01 mg/mL to 2 mg/mL tRNA, 1-100 mg/mL folinic acid and 10 mM to 200 mM buffer.

The step (b) is carried out at temperatures ranging from 20° C. to 50° C., preferably 30-40° C. for 1-24 hr. preferably 1-3 hr.

By eliminating the time-consuming steps required for the template preparation, without compromising the productivity, the present method should provide a useful platform for the rapid preparation of protein species for various analyses.

Through the simultaneous reduction of the exonucleolytic and endonucleolytic activities in the cell-free extract, the mRNA level was kept stable and protein synthesis was enhanced. Amounts of the synthesized proteins were sufficient for detection with conventional Coomassie blue dyes.

We expect the PCR-based cell-free protein synthesis system described in this specification to provide a versatile platform for the rapid and parallel synthesis of protein molecules.

The present invention will now be described in further detail by examples. It would be obvious to those skilled in the art that these examples are intended to be more concretely illustrative and the scope of the present invention as set forth in the appended claims is not limited to or by the examples.

### EXAMPLES

### Materials and Methods

### Materials

ATP, GTP, UTP, CTP, creatine phosphate, creatine kinase and E. coli total tRNA mixture were purchased from Roche Applied Science (Indianapolis, Ind.). L-[U-¹⁴C]leucine (11.9 GBq/mmol) was obtained from Amersham Biosciences (Uppsala, Sweden). Strain rne131 was obtained from Invitrogen under the narneof BL21-Star™ (DE3). All other reagents were purchased from Sigma. S30 extract were prepared as previously described [7] with minor modifications. Instead of its exogenous addition, T7 RNA polymerase was expressed during the cultivation of E. coli with IPTG induction (1.0 mM) at 0.5 OD₆₀₀. Cells were harvested 2 hours after induction and used for the preparation of the S30 extract.

### DNA Templates for Cell-Free Protein Synthesis

ORFs of five different proteins cloned into the plasmid pK7 [7] were used as the templates for the plasmid-based cell-free protein synthesis (the control reactions). For the experiments involving the PCR-based cell-free protein synthesis, the plasmids were amplified with a forward primer against the T7 promoter sequence. Depending on the experiment, a backward primer against the vector sequence immediately next to the stop codon or against the T7 terminator was used (Table 1). The PCR products were purified using a PCR-clean up kit (Promega) prior to their use for protein expression. The amount of PCR product was determined by spectrophotometry and agarose gel electrophoresis.

TABLE 1
Oligonucleotide primers used in this study

| | | |
|---|---|---|
| Oligonucleotide | Orientation | Sequence (5′ to 3′) |

| | | |
|---|---|---|
| T7P-15UP^{a} | Forward | TCGATCCCGCGAAATTAATACG |
| | | ACTCACTATAGG |
| ControL^{b} | Backward | CAGCTTCCTTTCGGGCTTTGTT |
| | | A |
| Poly-G^{c} | Backward | CCCCCCCCCCCCCCCCAGCTTC |
| | | CTTTCGGGCTTTGTTA |
| Mini-hairpin | Backward | CGCTTCGCGATGCTAGTTATTG |
| | | CT |
| T7 terminator | Backward | CAAAAAACCCCTCAAGACC |
| T7-5 | Backward | CAAAAAACCCGTTTAGGGGTTA |
| | | TGCTAGTTATTGCT |
| T7-7 | Backward | CAAAAAACCCCTGTTTAGAGGG |
| | | GTTATGCTAGTTATTGCT |
| T7-10 | Backward | CAAAAAACCCCTCAAGTTTAGC |
| | | CAAGGGGTTATGCTAGTTATTG |
| | | CT |
| T7-20 | Backward | CAAAAAACCCCCCCCCTCAAGA |
| | | CCCGTTTAGAGGCCCCAAGGGG |
| | | GGGGGTTATGCTAGTTATTGCT |
| T7-25 | Backward | CAAAAAACCCCCCCCCCCCCCT |
| | | CAAGACCCGTTTAGAGGCCCCA |
| | | AGGGGGGGGGGGGGGTTATGCT |
| | | AGTTATTGCT |
| T7-30 | Backward | CAAAAAACCCCCCCCCCCCCCC |
| | | CCCCTCAAGACCCGTTTAGAGG |
| | | CCCCAAGGGGGGGGGGGGGGGG |
| | | GGGTTATGCTAGTTATTGCT |

^{a}Fifteen extra base pairs were added to the 5′ end of the T7 promoter in pK7 for its optimal transcription by T7 RNA polymerase.
^{b}Upstream region of T7 terminator stem-loop in pK7 vector
^{c}Poly-G primer was designed to give fifteen Gs to transcript at 3′ terminus

T7-5, T7-7, T7-10, T7 terminator, T7-20, T7-25 and T7-30 primers in Table 1 were used for generating termination sequences set forth in SEQ ID NOs:12-18, respectively.

### Cell-Free Protein Synthesis and Analysis

The standard reaction mixture for the coupled transcription/translation consisted of the following components in a total volume of 15 μl: 57 mM Hepes/KOH pH 8.2, 1.2 mM ATP, 0.85 mM each of GTP, UTP and CTP, 1.7 mM dithiothreitol, 80 mM ammonium acetate, 0.17 mg/ml E. coli total tRNA mixture (from strain MRE 600), 34 mg/ml 1-5-formyl-5,6,7,8-tetrahydrofolic acid (folinic acid), 1.5 mM each of the unlabeled amino acids, 0.3 U/ml creatine kinase, 67 mM creatine phosphate, and 4 μl S30 extract. Depending on the experiment, 0.1 μg plasmid or 0.5 μg PCR-products were used as the templates to direct the protein synthesis. The synthesis reactions were conducted in a waterbath at 37° C. for 2 hours. The synthesized protein was quantified by measuring the TCA-precipitable radioactivities as described in a previous report [7]. Molecular weight of the expressed protein was confirmed a 13% Tricine-SDS-polyacrylamide gel [8].

In order to confirm the biological activity of the expressed protein, the fluorescence of the synthesized EGFP was measured using a fluorescence spectrometer (excitation, 488 nm; emission, 507 nm) after 1:100 dilution in assay buffer (100 mM HEPES-KOH pH 7.6, 14 mM magnesium acetate, 60 mM potassium acetate, 0.5 mM dithiothreitol).

Analysis of mRNA Stability

5 μl samples were withdrawn during the incubation of the reaction mixture and immediately mixed with equal volumes of RNA protect bacterial reagent (Qiagen). The mRNA in each sample was then extracted and purified using a commercial kit (RNeasy Mini, Qiagen). The size of the isolated mRNA was analyzed on a 2% formaldehyde agarose gel.

### RESULTS AND DISCUSSION

Enabling the direct expression of PCR-amplified genes in a cell-free protein synthesis system would greatly increase the throughput of the protein preparation process by eliminating the requirements for cloning and plasmid amplification, as well as the procedures cultivating the cells. However, linear DNAs, such as PCR products, are highly susceptible to degradation by the intracellular exonucleases of Escherichia coli that remain active in the cell-free extracts [9]. For this reason, compared to conventional reactions using plasmid templates, the productivity of cell-free protein synthesis from PCR-amplified DNA is very low. The stabilization of linear DNA in a cell-free extract represents quite a challenge, since the presence of exonucleolytic DNase activity is essential for the survival and growth of the cells. However, since the sequence information of DNA is amplified and transformed into mRNA molecules before it is translated, the problem of template depletion can be overcome if the transcribed mRNA remains intact during the cell-free protein synthesis.

Effects of 3′-UTR Sequences on mRNA Stability and the Efficiency of Cell-Free Protein Synthesis Using PCR-Amplified Templates

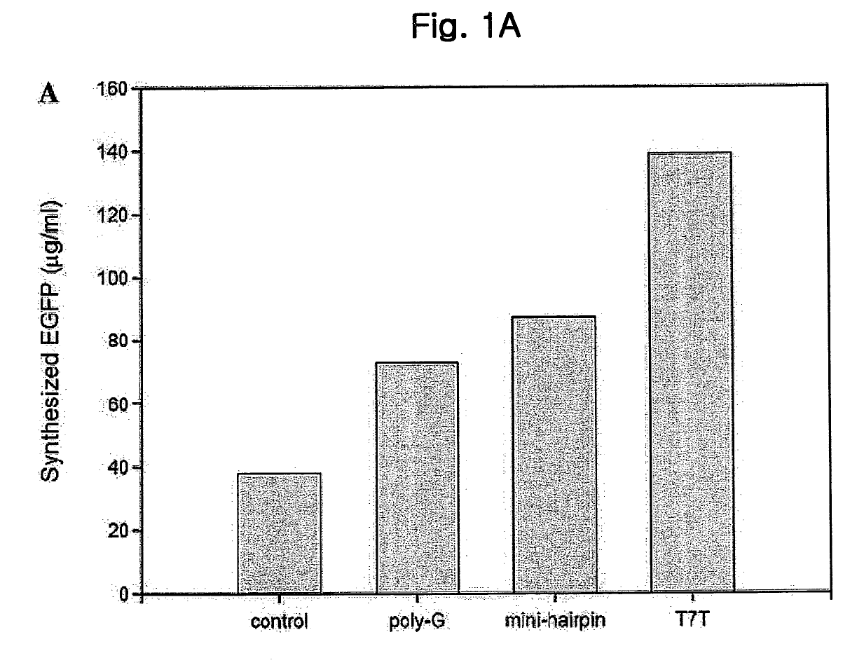

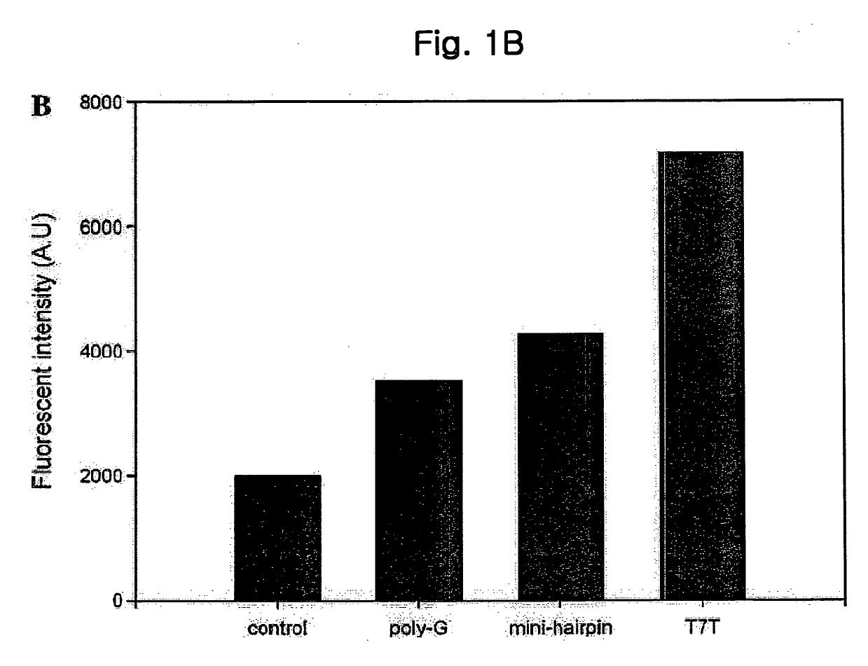

Lisitsky and Schuster reported that the chemical and functional half-life of mRNA is improved in the presence of a 3′ poly (G) tail by virtue of its improved resistance against PNPase activity [10]. In addition, Lee and Cohen showed that the addition of a poly(G) tail enhanced the mRNA stability and protein expression during cell-free protein synthesis [6]. In our experiments involving the expression of EGFP from DNA templates prepared by PCR, the protein expression was also substantially enhanced when 15 guanosine residues were added to the mRNA sequence of EGFP (FIG. 1).

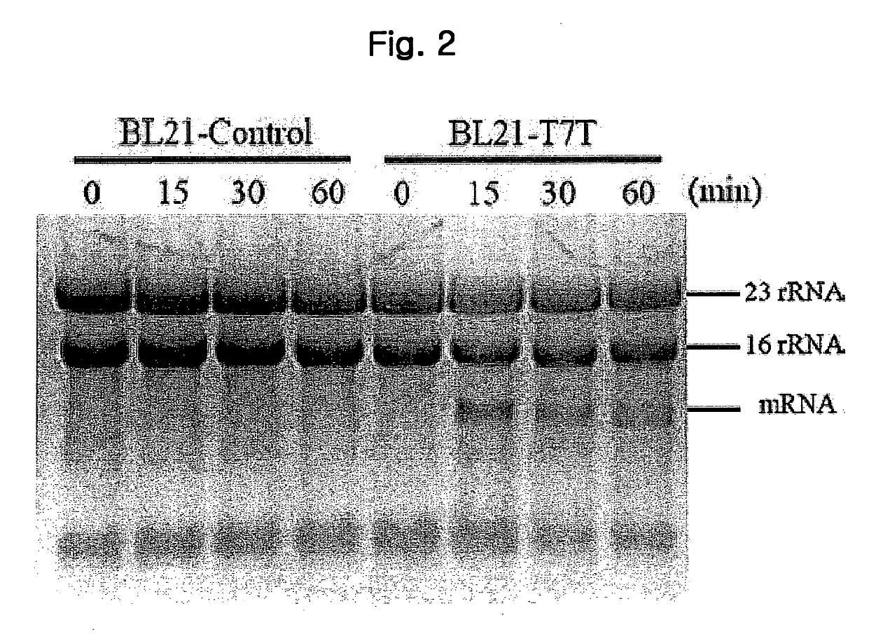

As another sequence element that can be used to enhance the stability of mRNA, it has been reported that the secondary structures at the 3′ end of the mRNA molecule serve as barriers to the action of 3′→5′ exonucleases [11]. Based on this information, we examined the effects on the mRNA stability and protein synthesis of two stem-loop forming sequences, viz. the mini-hairpin sequence [12] and the sequence of the T7 transcription terminator [17]. These stem-loop sequences were added downstream of the stop codon using the backward primers given in Table 1. The presence of either of these stem-loop structures was found to substantially increase the stability of the transcript and to stimulate the protein expression more effectively than the poly (G) tail sequence (FIG. 1A). Especially, the addition of the T7 terminator sequence led to a substantial increase in protein synthesis. Although the presence of the T7 terminator sequence is not essential for the termination of transcription in the present reaction format (linear DNAs can be run-off transcribed), an approximately 4-fold increase in protein yield was observed in its presence. The fluorescence of the reaction samples was found to be proportional to the amount of expressed EGFP (FIG. 1B). Formaldehyde-agarose gel analysis of the time-course samples indicated that the increase in protein synthesis is related to the enhanced mRNA levels (FIG. 2). Whereas the transcripts from the control DNA were not even detected with the present method, clear bands corresponding to the T7 terminator-containing mRNA were observed for at least an hour.

In the separate experiment of in vitro transcription, it was observed that the efficiency of transcription was not significantly affected by the presence or absence of the T7 terminator sequence (data not shown). Therefore, we assumed that the extended maintenance of the mRNA level in the presence of the T7 terminator sequence could be attributed to the enhanced stability of the transcribed mRNAs.

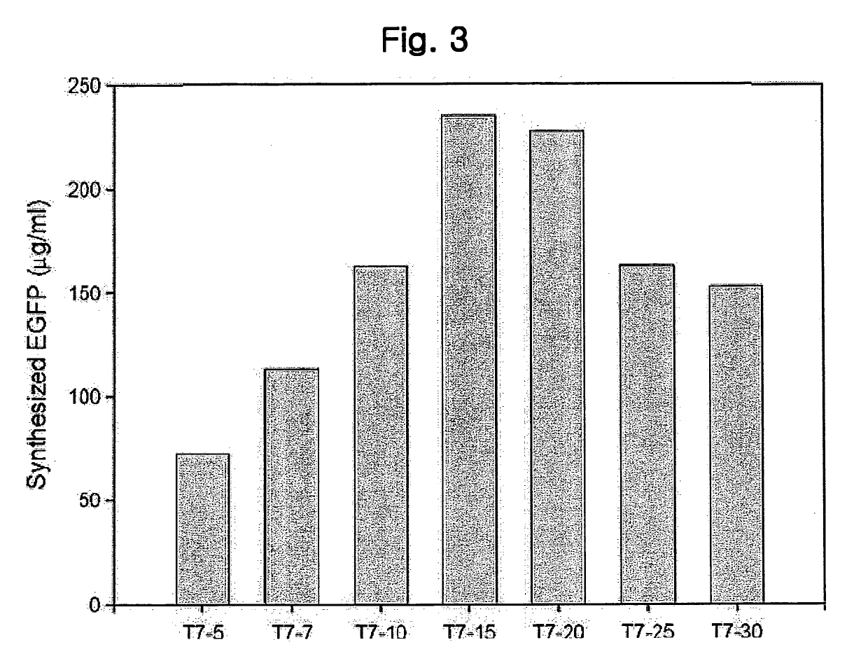

It appears that the effectiveness of the stem-loop sequence is dependent upon the length of the tightly base-paired stem structure. When the pairing bases were sequentially removed from the native sequence of the T7 terminator, the level of protein expression showed a gradual decrease depending on the number of base-pairs that were removed. However, the addition of extra base pairs did not afford any further enhancement of the protein expression, and the natural sequence of the T7 terminator gave the optimal result (FIG. 3).

Cell-Free Protein Synthesis in a Cell Extract Prepared from RNase E Mutant Strain

As described above, the presence of nuclease-resistant sequences at the 3′ end of the mRNA significantly increased the level of cell-free protein synthesis obtained from a linear, PCR-amplified DNA template. However, the amount of synthesized protein was still far less than that which can be obtained from plasmid templates. While approximately 140 μg/ml of EGFP was produced from the PCR-products encoding the T7 terminator, approximately 380 μg/ml of protein was obtained when the identical sequence was cloned into a plasmid and expressed under the same reaction conditions. The time-course comparison of the mRNA levels indicated that, even in the presence of the terminator sequence, the overall level of mRNA in the PCR-based reaction was also still lower than that in the plasmid-based reaction (data not shown). Therefore, it appears that the protection of the 3′ end is not sufficient to keep the mRNA intact in an efficient manner.

In Escherichia coli cells, mRNA degradation is initiated by endonucleolytic cleavage and proceeds with the subsequent 3′→5′ exonucleolytic digestion of the fragments [13]. Therefore, the stabilizing effect afforded by the 3′ end protection would be expected to be limited, as long as the extract retains endonucleolytic activities. Based on this assumption, we expected the stabilizing effect of the T7 terminator sequence to be fully exhibited when the endonucleolytic cleavage of the mRNA was properly prevented as well. RNase E is the most potent endonuclease of Escherichia coli and is responsible for the degradation of most mRNAs [14, 15]. Previously, Lopez et al reported that the removal of the C-terminal half of RNase E (rne 131) inhibits its ability to degrade mRNA without affecting cell growth [16]. Therefore, cell-free extract was prepared from a strain that has the same genetic background as BL21(DE3), but carries the rne 131 mutation (hereafter referred to as rne 131-extract).

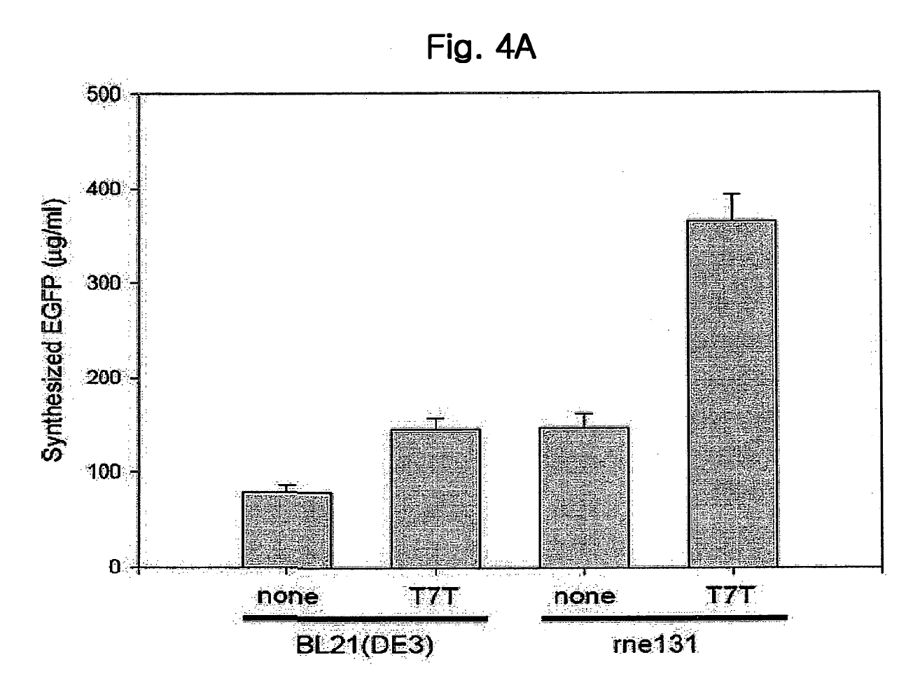

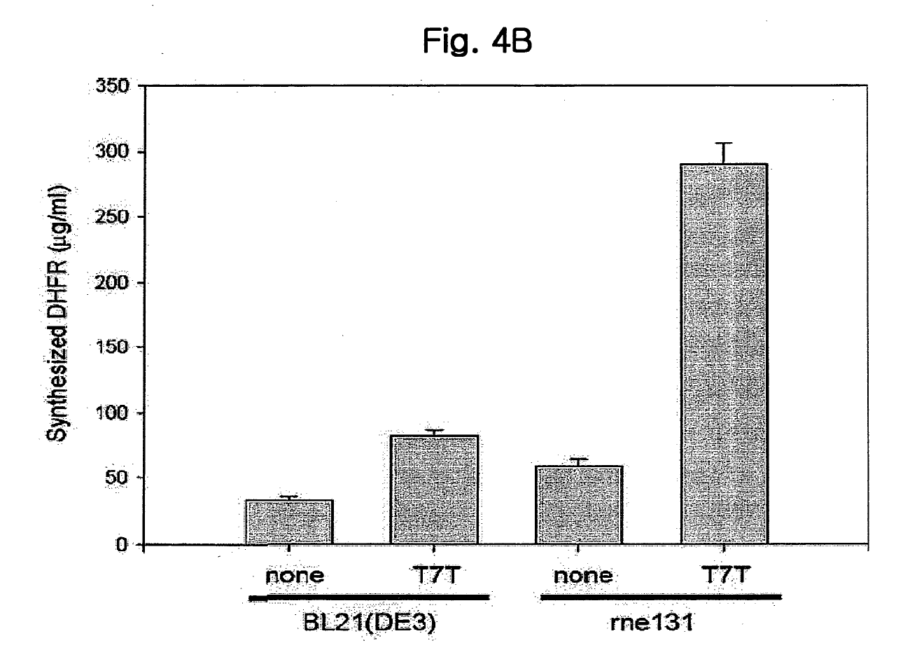

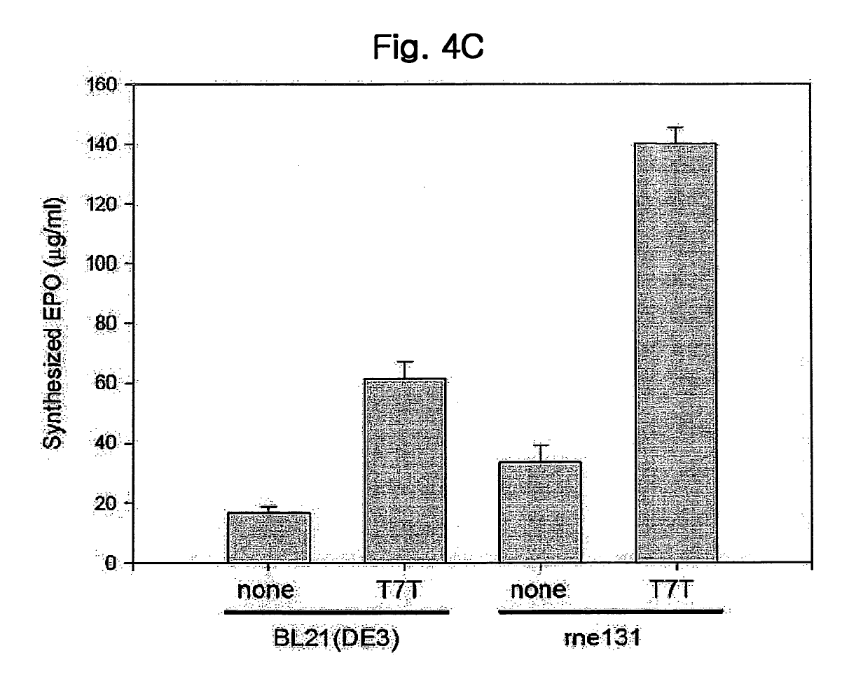

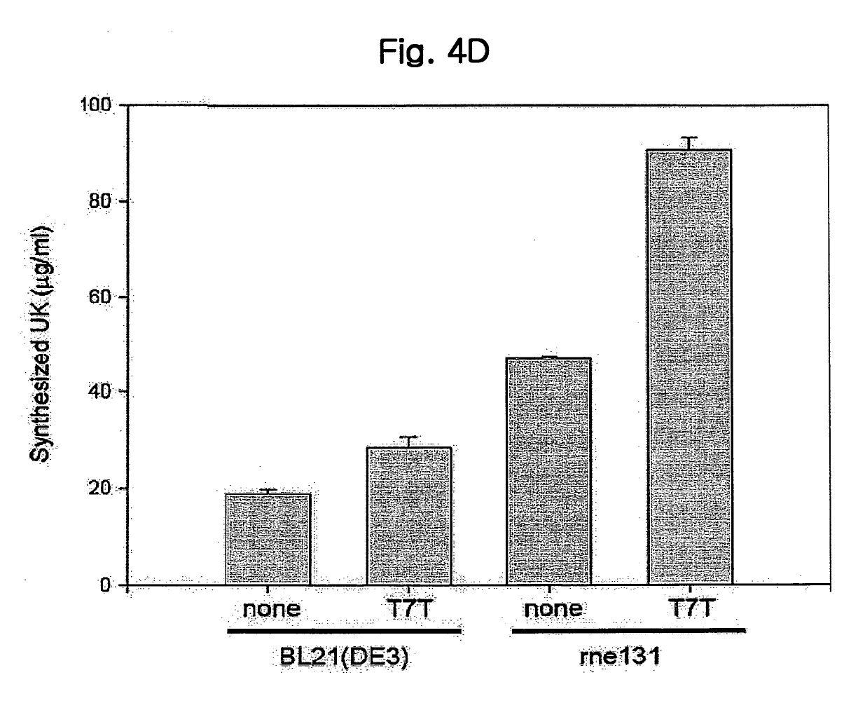

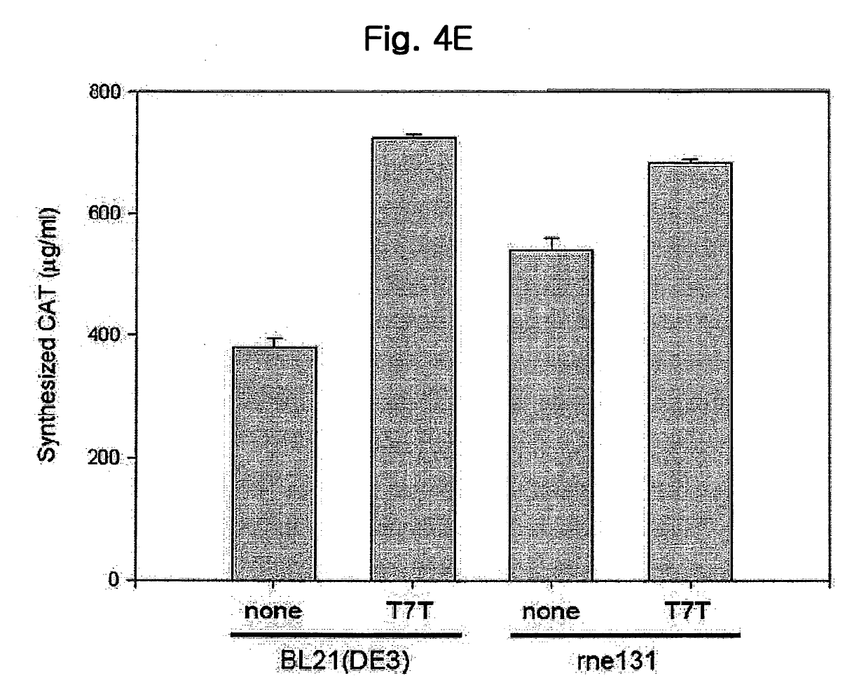

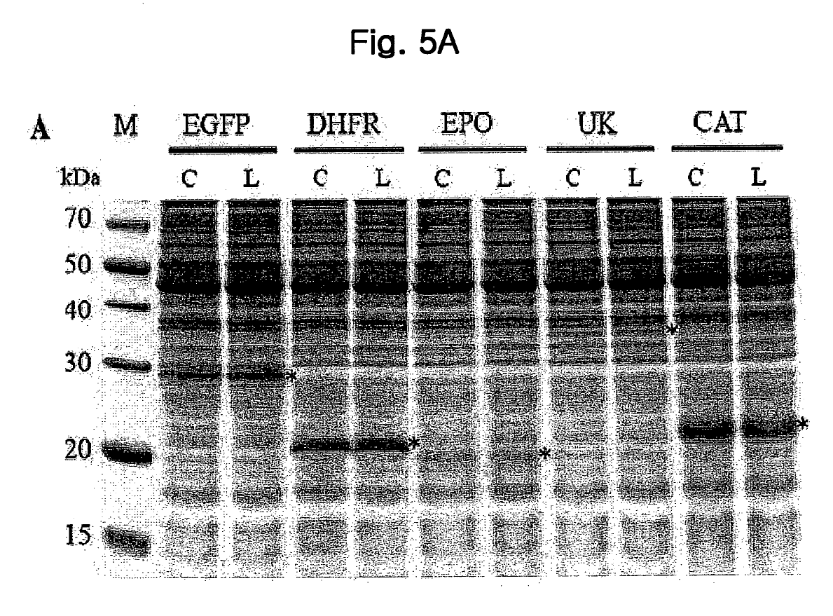

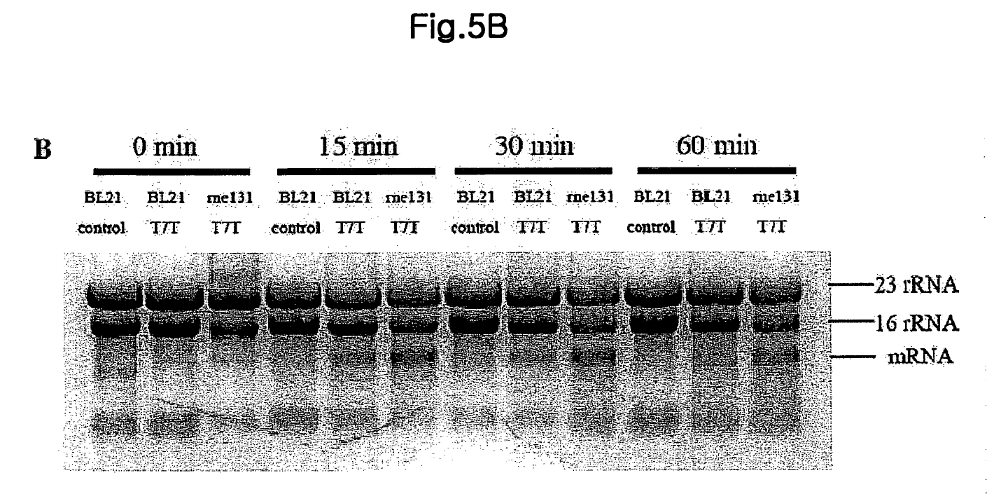

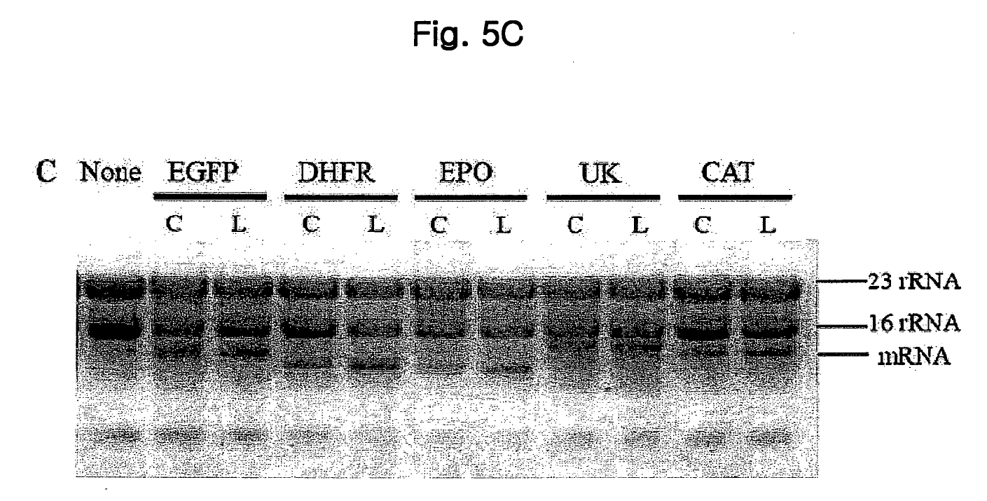

Five independent genes [erythropoietin (EPO), dihydrofolate reductase (DHFR), urokinase (UK), enhanced green fluorescent protein (EGFP), and chloroamphenicol acetyltransferase (CAT)] were PCR-amplified with the primers given in Table 1. When the sequences amplified without the T7 terminator were used to direct the cell-free protein synthesis, a 50-200% increase in protein synthesis was observed in the rne 131 extract (FIG. 4) as compared to that observed with the standard extract prepared from BL21 (DE3). More importantly, protein synthesis was strikingly improved when the T7 terminator-containing PCR products were expressed in the rne 131 extract. As is summarized in Table 2 and FIG. 5A, when expressed in the rne 131 extract, the amount of proteins produced from the T7 terminator-containing PCR products was comparable to that observed in the conventional reactions based on plasmid templates.

TABLE 2
Comparison of expression level between plasmid and PCR-amplified
DNA in cell extract prepared from rne 131 mutant

| | Average productivity ± SD | |
|---|---|---|
| Genes* | (μg/ml) | Relative level of expression** |
| EGFP | 364.5 ± 20.1 | 0.96 |
| DHFR | 290.0 ± 11.7 | 0.84 |
| EPO | 139.9 ± 3.9 | 1.03 |
| UK | 90.8 ± 1.7 | 0.94 |
| CAT | 685.1 ± 3.3 | 1.01 |

*EGFP: enhanced green fluorescence protein, DHFR: dihydrofolate reductase, EPO: erythropoietin, UK: urokinase, CAT: chloramphenicol acetyltransferase.
**Level of expression using the PCR product as a template as compared to that observed using the plasmid (plasmid expression level = 1)

Time-course analysis of the mRNA transcribed from the DNA sequence of DHFR demonstrated that the stability of mRNA was remarkably enhanced through the use of rne 131 extract and the T7 terminator sequence (FIG. 5B). In addition, for all of the reactions with five different proteins, residual amounts of the intact mRNA after a 30 min incubation were comparable between the reactions utilizing the plasmids and PCR-amplified DNAs (FIG. 5C).

### CONCLUSION

In the present study, we demonstrated that the productivity of PCR-based cell-free protein synthesis can be made comparable to that of the conventional reactions using plasmid templates. Through the simultaneous reduction of the exonucleolytic and endonucleolytic activities in the cell-free extract, the mRNA level was kept stable and protein synthesis was enhanced. Amounts of the synthesized proteins were sufficient for detection with conventional Coomassie blue dyes.

We expect the PCR-based cell-free protein synthesis system described in this specification to provide a versatile platform for the rapid and parallel synthesis of protein molecules.

Having described a preferred embodiment of the present invention, it is to be understood that variants and modifications thereof falling within the spirit of the invention may become apparent to those skilled in this art, and the scope of this invention is to be determined by appended claims and their equivalents.

### References

[1] F. Katzen, G. Chang, and W. Kudlicki, The past, present and future of cell-free protein synthesis, Trends Biotechnol. 23 (2005) 150-156.
[2] J. M. Betton, High throughput cloning and expression strategies for protein production, Biochimie 86 (2004) 601-605.
[3] R Braun, and J. LaBaer, High throughput protein production for functional proteomics, Trends Biotechnol. 21 (2003) 383-388.
[4] S. A. Lesley, M. A. Brow, and R. R. Burgess, Use of in vitro protein synthesis from polymerase chain reaction-generated templates to study interaction of Escherichia coli transcription factors with core RNA polymerase and for epitope mapping of monoclonal antibodies, J. Biol. Chem. 266 (1991) 2632-2638.
[5] J. M. Pratt, G. J. Boulnois, V. Darby, E. Orr, E. Wahle, and I. B. Holland, Identification of gene products programmed by restriction endonuclease DNA fragments using an E. coli in vitro system, Nucleic Acids Res. 9 (1981) 4459-4474.
[6] K. Lee, and S. N. Cohen, Effects of 3′ terminus modifications on mRNA functional decay during in vitro protein synthesis, J. Biol. Chem. 276 (2001) 23268-23274.
[7] D. M. Kim, T Kigawa, C. Y Choi, and S. Yokoyama, A highly efficient cell-free protein synthesis system from Escherichia coli, Eur. J. Biochem. 239 (1996) 881-886.
[8] H. Schagger, and G. von Jagow, Tricine-sodium dodecyl sulfate-polyacrylamide gel electrophoresis for the separation of proteins in the range from 1 to 100 kDa, Anal. Biochem. 166 (1987) 368-379.
[9] K. Sitaraman, D. Esposito, G. Klarmann, S. F. Le Grice, J. L. Hartley, and D. K. Chatterjee, A novel cell-free protein synthesis system, J. Biotechnol. 110 (2004) 257-263.
[10] I. Lisitsky, and G. Schuster, Preferential degradation of polyadenylated and polyuridinylated RNAs by the bacterial exoribonuclease polynucleotide phosphorylase, Eur. J. Biochem. 261 (1999) 468-474.
[11] M. J. Stern, G. F. Ames, N. H. Smith, E. C. Robinson, and C. F. Higgins, Repetitive extragenic palindromic sequences: a major component of the bacterial genome, Cell 37 (1984) 1015-1026.
[12] I. Hirao, S. Yoshizawa, and K. Miura, Stabilization of mRNA in an Escherichia coli cell-free translation system, FEBS Lett. 321 (1993) 169-172.
[13] M. Grunberg-Manago, Messenger RNA stability and its role in control of gene expression in bacteria and phages, Annu. Rev. Genet. 33 (1999) 193-227.
[14] G. A. Coburn, and G. A. Mackie, Degradation of mRNA in Escherichia coli: an old problem with some new twists, Prog Nucleic Acid Res Mol Biol 62 (1999) 55-108.
[15] D. R Nierlich, and G. J. Murakawa, The decay of bacterial messenger RNA, Prog. Nucleic Acid Res. Mol. Biol. 52 (1996) 153-216.
[16] R J. Lopez, I. Marchand, S. A. Joyce, and M. Dreyfus, The C-terminal half of RNase E, which organizes the Escherichia coli degradosome, participates in mRNA degradation but not rRNA processing in vivo, Mol. Microbiol. 33 (1999) 188-199.
[17] H. Nakano, et al., Efficient coupled transcription/translation from PCR template by a hollow-fiber membrane bioreactor, Biotechnol. Bioeng. 64 (1999) 194-199

What is claimed is:

1. A method for synthesizing a protein in a cell-free synthesis system, which comprises the steps of:
(a) amplifying a DNA molecule encoding the protein to prepare linear DNA molecules with a stem and loop sequence at their terminus, wherein the stem and loop sequence comprises a nucleotide sequence inducing the formation of a stem and loop structure at the 3′-end of transcripts of the amplified DNA molecules to prevent degradation of the transcripts; and
(b) contacting the amplified DNA molecules to a lysate of cells with deficient endoribonuclease E activity, thereby producing the protein in the cell-free synthesis system.

2. The method according to claim 1, wherein the stem of the stem and loop structure has 5-30 base pairs.

3. The method according to claim 2, wherein the stem of the stem and loop structure has 15-20 base pairs.

4. The method according to claim 1, wherein the stem of the stem and loop structure is a transcription termination sequence.

5. The method according to claim 1, wherein the stem and loop structure has the nucleotide sequence selected from the group consisting of SEQ ID NOs:12-19.

6. The method according to claim 5, wherein the transcription termination sequence is selected from the group consisting of SEQ ID NOs:14-18.

7. The method according to claim 6, wherein the transcription termination sequence is set forth in SEQ ID NOs:15 or 16.

8. The method according to claim 1, wherein the cells with deficient endoribonuclease E activity is a bacterial strain with deficient endoribonuclease E activity.

9. The method according to claim 1, wherein the cells with deficient endoribonuclease E activity is E. coli with deficient endoribonuclease E activity.

10. The method according to claim 1, wherein the cells with deficient endoribonuclease E activity is E. coli rne 131 strain with deficient endoribonuclease E activity.


SEQUENCE LISTING (19 sequences)

SEQ ID NO: 1 - DNA, 34 bp, Artificial Sequence
  T7P-15UP primer
tcgatcccgcgaaattaatacgactcactatagg

SEQ ID NO: 2 - DNA, 23 bp, Artificial Sequence
  control primer
cagcttcctttcgggctttgtta

SEQ ID NO: 3 - DNA, 38 bp, Artificial Sequence
  Poly-G primer
ccccccccccccccccagcttcctttcgggctttgtta

SEQ ID NO: 4 - DNA, 24 bp, Artificial Sequence
  Mini-hairpin primer
cgcttcgcgatgctagttattgct

SEQ ID NO: 5 - DNA, 19 bp, Artificial Sequence
  T7 terminator primer
caaaaaacccctcaagacc

SEQ ID NO: 6 - DNA, 36 bp, Artificial Sequence
  T7-5 primer
caaaaaacccgtttaggggttatgctagttattgct

SEQ ID NO: 7 - DNA, 40 bp, Artificial Sequence
  T7-7 primer
caaaaaacccctgtttagaggggttatgctagttattgct

SEQ ID NO: 8 - DNA, 46 bp, Artificial Sequence
  T7-10 primer
caaaaaacccctcaagtttagccaaggggttatgctagttattgct

SEQ ID NO: 9 - DNA, 66 bp, Artificial Sequence
  T7-20 primer
caaaaaaccccccccctcaagacccgtttagaggccccaagggggggggttatgctagttattgct

SEQ ID NO: 10 - DNA, 76 bp, Artificial Sequence
  T7-25 primer
caaaaaacccccccccccccctcaagacccgtttagaggccccaaggggggggggggggttatgctagttattgct

SEQ ID NO: 11 - DNA, 86 bp, Artificial Sequence
  T7-30 primer
caaaaaaccccccccccccccccccctcaagacccgtttagaggccccaagggggggggggggggggggttatgctagttattgct

SEQ ID NO: 12 - DNA, 28 bp, Artificial Sequence
  T7-5 stem-loop synthetic
ctagcataacccctaaacgggttttttg

SEQ ID NO: 13 - DNA, 32 bp, Artificial Sequence
  T7-7 stem-loop synthetic
ctagcataacccctctaaacaggggttttttg

SEQ ID NO: 14 - DNA, 38 bp, Artificial Sequence
  T7-10 stem-loop synthetic
ctagcataaccccttggctaaacttgaggggttttttg

SEQ ID NO: 15 - DNA, 48 bp, Artificial Sequence
  T7-15 stem-loop synthetic
ctagcataaccccttggggcctctaaacgggtcttgaggggttttttg

SEQ ID NO: 16 - DNA, 58 bp, Artificial Sequence
  T7-20 stem-loop synthetic
ctagcataacccccccccttggggcctctaaacgggtcttgagggggggggttttttg

SEQ ID NO: 17 - DNA, 68 bp, Artificial Sequence
  T7-25 stem-loop synthetic
ctagcataaccccccccccccccttggggcctctaaacgggtcttgaggggggggggggggttttttg

SEQ ID NO: 18 - DNA, 78 bp, Artificial Sequence
  T7-30 stem-loop synthetic
ctagcataacccccccccccccccccccttggggcctctaaacgggtcttgagggggggggggggggggggttttttg

SEQ ID NO: 19 - DNA, 16 bp, Artificial Sequence
  mini-hairpin stem-loop synthetic
ctagcatcgcgaagcg